(19) United States
(12) Patent Application Publication
(10) Pub. No.: US 2007/0142457 A1
(43) Pub. Date: Jun. 21, 2007

(54) Crystalline forms of docetaxel and processes for their preparation

(76) Inventors: Alessandro Pontiroli, Maria della Versa (PV) (IT); Marco Villa, Milano (IT); Judith Aronhime, Rehovot (IL); Sigalit Levi, Modi'in (IL)

Correspondence Address: KENYON & KENYON LLP, ONE BROADWAY, NEW YORK, NY, 10004, US

(21) Appl. No.: 11/580,626

(22) Filed: Oct. 12, 2006

Related U.S. Application Data
(60) Provisional application No. 60/725,321, filed on Oct. 12, 2005.
(60) Provisional application No. 60/726,647, filed on Oct. 17, 2005.
(60) Provisional application No. 60/736,870, filed on Nov. 16, 2005.

(51) Int. Cl. A61K 31/337; C07D 305/12

(52) U.S. Cl. 514/449; 549/510

(57) ABSTRACT

Novel forms of crystalline docetaxel are provided, as well as pharmaceutical compositions, and methods of treatment. Novel processes for making crystalline docetaxel are also provided.

### CROSS-REFERENCE TO RELATED APPLICATIONS

This application claims the benefit of U.S. Provisional Application Ser. No. 60/725,321, filed Oct. 12, 2005; U.S. Provisional Application Ser. 60/726,647, filed Oct. 17, 2005; and U.S. Provisional Application Ser. No. 60/736,870, filed Nov. 16, 2005. These applications are incorporated herein by reference.

### FIELD OF THE INVENTION

The invention encompasses the solid state chemistry of docetaxel. In certain embodiments, novel crystalline forms of docetaxel are provided, as well as methods for the preparation thereof.

### BACKGROUND OF THE INVENTION

Docetaxel is an antineoplastic agent belonging to the taxoid family. Docetaxel is a semi-synthetic anticancer drug deriving from chemical modification of a taxane core (named 10-deacetylbaccatin III or 10-DAB) extracted from leaves and bark of European or Indian yew trees. The chemical name for docetaxel is: (2R,3S)—N-carboxy-3-phenylisoserine, N-tert-butyl ester, 13-ester with 5(β)-20-epoxy-1,2(α), 4,7(beta), 10(β), 13(α)-hexahydroxytax-11-en-9-one 4-acetate 2-benzoate, trihydrate. Docetaxel is represented by the formula:

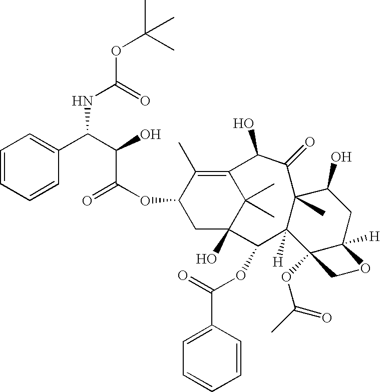

Docetaxel is a white to off-white powder with an empirical formula of C₄₃H₅₃NO₁₄.3H₂O, and a molecular weight of 861.9.

Docetaxel is marketed under the name TAXOTERE® as a concentrated injection. TAXOTERE® injection is a clear yellow to brownish-yellow viscous solution. TAXOTERE® injection is available in single-dose vials containing 20 mg (0.5 ml) or 80 mg (0.2 ml) docetaxel (anhydrous).

The occurrence of different crystal forms (polymorphism) is a property of some molecules and molecular complexes. A single molecule, or a salt complex, may give rise to a variety of solids having distinct physical properties like melting point, X-ray diffraction pattern, infrared absorption fingerprint and NMR spectrum. The crystalline form may give rise to thermal behavior different from that of the amorphous material or another crystalline form. Thermal behavior is measured in the laboratory by such techniques as capillary melting point, thermogravimetric analysis (“TGA”) and differential scanning calorimetry (“DSC”) and can be used to distinguish some polymorphic forms from others. The differences in the physical properties of different crystalline forms result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. These properties can be influenced by controlling the conditions under which the salt is obtained in solid form.

Exemplary solid state physical properties include the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment.

The solid-state characterization of docetaxel is reported in J. Phys. IV France 11 (2001). The article discloses a stochiometric hydrate containing three water molecules per molecule (trihydrate) of drug substance. It also reports that “under a nitrogen steam, dynamic dry atmosphere (0% RH), or under heat, docetaxel is dehydrated and forms an anhydrous crystal phase.”

U.S. Pat. No. 6,022,985, discloses, according to its abstract, a “process for the preparation of 4-acetoxy-2.alpha.-benzoyloxy-5.beta.,20-epoxy-1,7.beta., 10.beta.-trihydroxy-9-oxo-tax-11-en-13.alpha.-yl (2R,3S)-3-tert-butoxycarbonylamino-2-hydroxy-3-phenylpropionate trihydrate, characterized in that the 4-acetoxy-2.alpha.-benzoyloxy-5.beta.,20-epoxy-1,7.beta.,10.beta.-trihydroxy-9-oxo-tax-11-en-13.alpha.-yl (2R,3S)-3-tert-butoxycarbonylamino-2-hydroxy-3-phenylpropionate is crystallized from a mixture of water and an aliphatic alcohol containing 1 to 3 carbon atoms, and then the product obtained is dried under defined conditions of temperature, pressure and humidity.”

U.S. Pat. No. 6,838,569, discloses, according to its abstract, “a process for converting paclitaxel or docetaxel to the respective trihydrate . . . comprises dissolving either paclitaxel or docetaxel in a mixture of alkane and chlorinated alkane to provide a crude product of 65-75% assay and dissolving the crude product in an alkyl ketone, followed by addition of an alkane to provide a product . . . dissolving the product of increased chromatographic purity in an aliphatic nitrile, with addition of water to precipitate taxane trihydrate.”

The discovery of new forms of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. Additional polymorphic forms may further help in determination of polymorphic content of a batch of an active pharmaceutical ingredient, for example, by providing a useful reference standard for XRD instruments.

### SUMMARY OF THE INVENTION

In one embodiment, the present invention encompasses a crystalline form of docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹).

Another embodiment of the invention encompasses a process for preparing crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹), including crystallizing the crystalline docetaxel from a mixture of methyl isobutyl ketone (MIBK) and an organic antisolvent.

Another embodiment of the invention encompasses processes for preparing a crystalline docetaxel form characterized by main X-ray powder diffraction peaks at about 8.0, 11.3, 12.5, 15.5 and 16.9±0.2 degrees two-theta including precipitating the crystalline docetaxel form from a mixture of a solvent and an organic antisolvent, wherein the solvent is selected from the group consisting of: acetone and ethylacetate, acetone and t-butanol, tetrahydrofuran (THF), ethyl acetate, tert-butanol, ethanol, and mixtures thereof.

In one embodiment, the invention encompasses a pharmaceutical composition containing a pharmaceutically effective amount of the crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹), and at least one pharmaceutically acceptable excipient.

Another embodiment of the invention encompasses a process of preparing a pharmaceutical composition including the step of combining crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹), or a solution prepared from the crystalline docetaxel, with a pharmaceutically acceptable carrier.

Yet another embodiment of the invention encompasses a pharmaceutical composition containing a therapeutically effective amount of crystalline docetaxel prepared according to the processes of the present invention, and at least one pharmaceutically acceptable excipient.

Another embodiment of the invention encompasses the use of crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹) in the manufacture of a medicament.

Yet another embodiment of the invention encompasses a method of treating a proliferative disorder, such as cancer, in a mammal, such as a human, including administering to the mammal a pharmaceutically acceptable amount of a pharmaceutical composition containing or prepared from crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹).

### BRIEF DESCRIPTION OF THE FIGURES

FIG. 1 illustrates a characteristic PXRD pattern of the crystalline docetaxel of the invention.

FIG. 2 illustrates a characteristic FTIR pattern of the crystalline docetaxel of the invention.

FIG. 3 illustrates a characteristic DSC thermogram of the crystalline docetaxel of the invention.

FIG. 4 illustrates a characteristic PXRD pattern of crystalline docetaxel characterized by main X-ray powder diffraction peaks at about 8.0, 11.3, 12.5, 15.5 and 16.9±0.2 degrees two-theta.

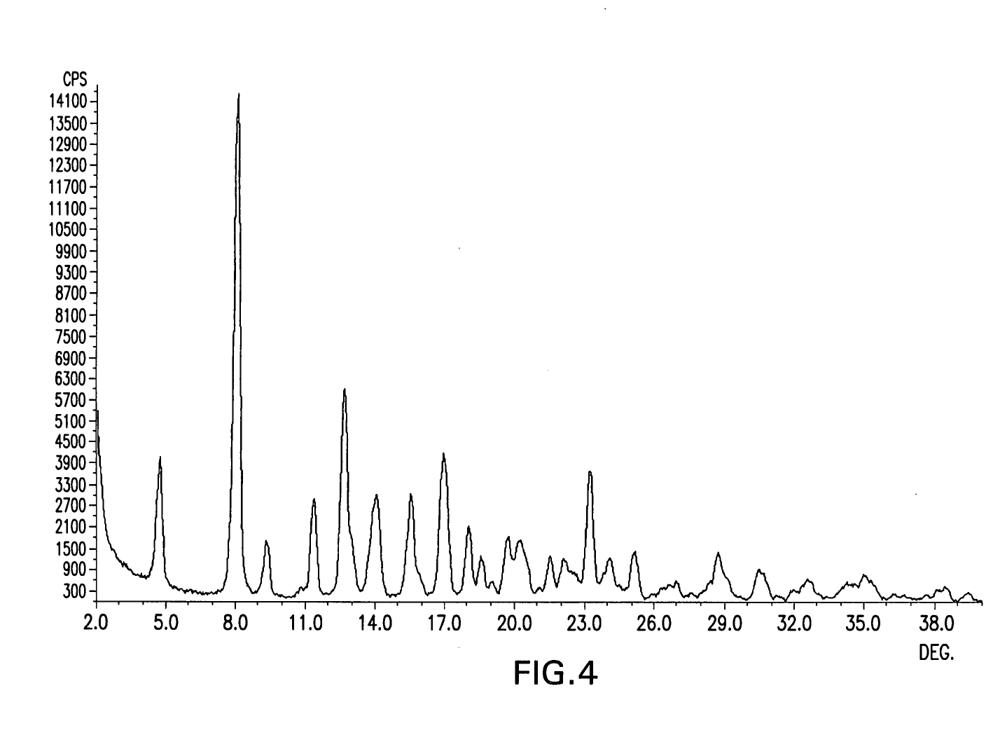

FIG. 5 illustrates a characteristic DSC thermogram of crystalline docetaxel characterized by main X-ray powder diffraction peaks at about 8.0, 11.3, 12.5, 15.5 and 16.9±0.2 degrees two-theta.

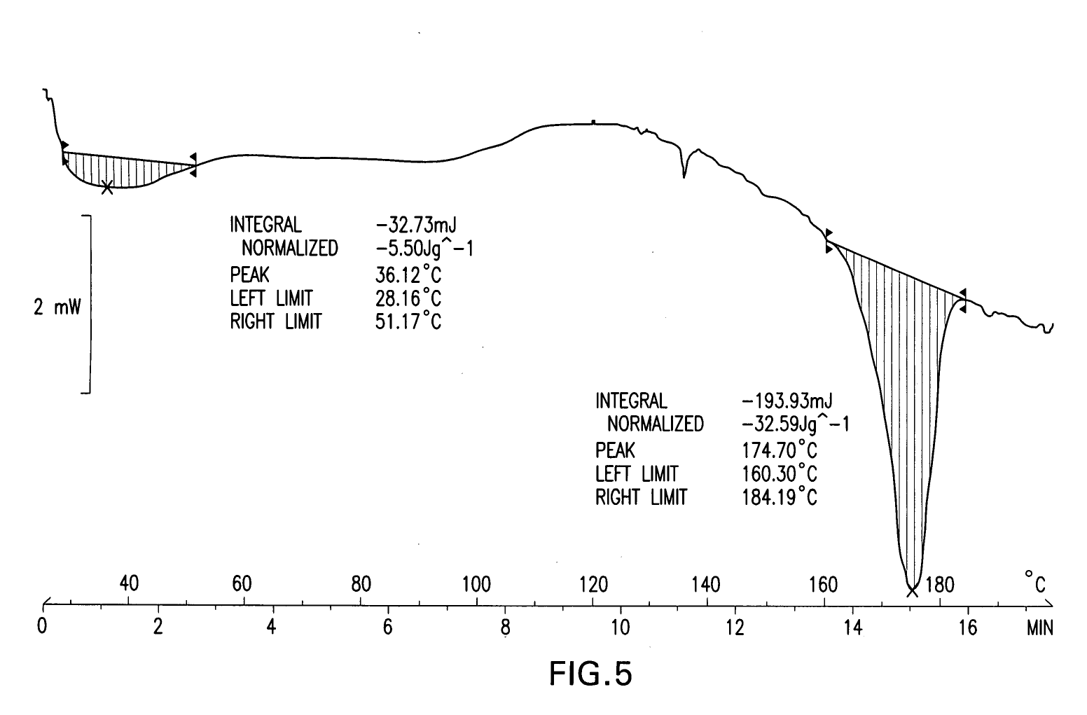

### DETAILED DESCRIPTION OF THE INVENTION

The present invention provides polymorphic forms of docetaxel and methods of making these. The docetaxel polymorph of the invention exhibits greater solubility than docetaxel trihydrate.

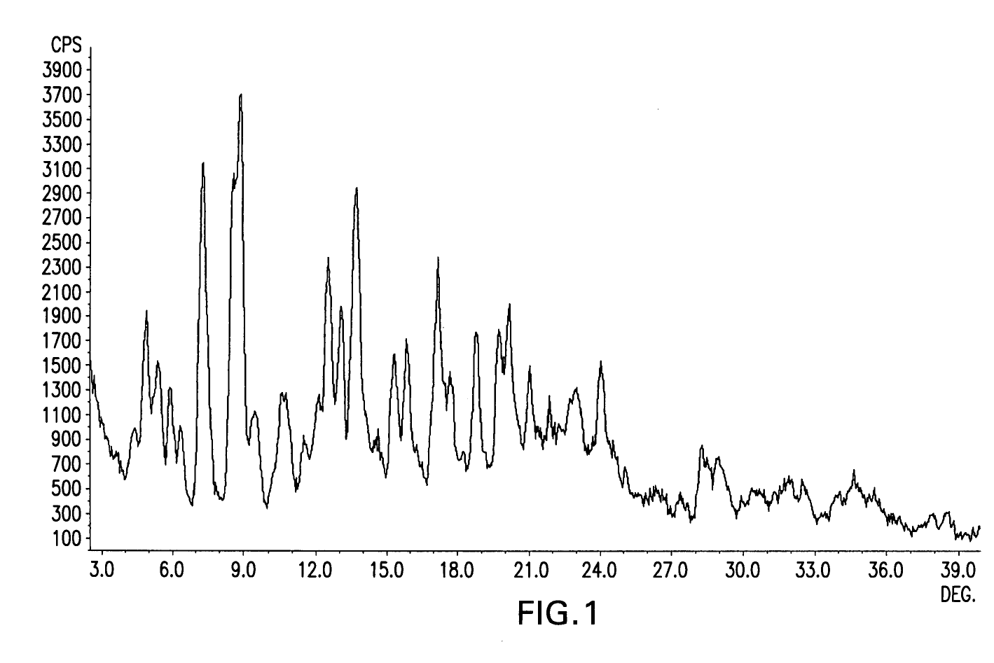

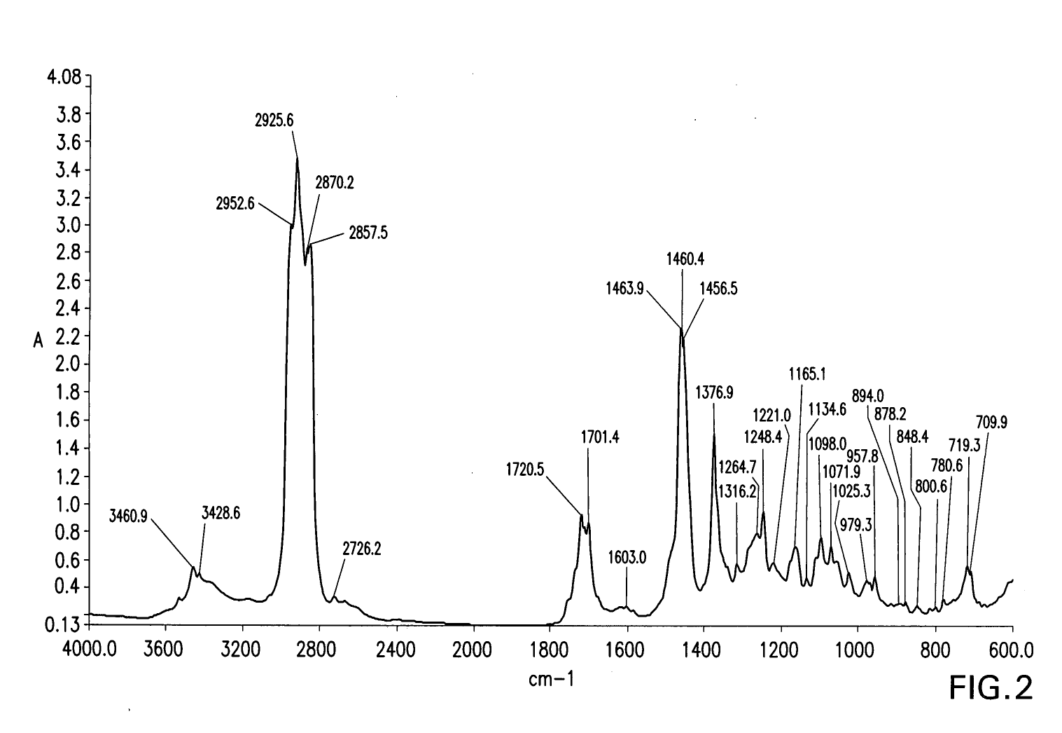

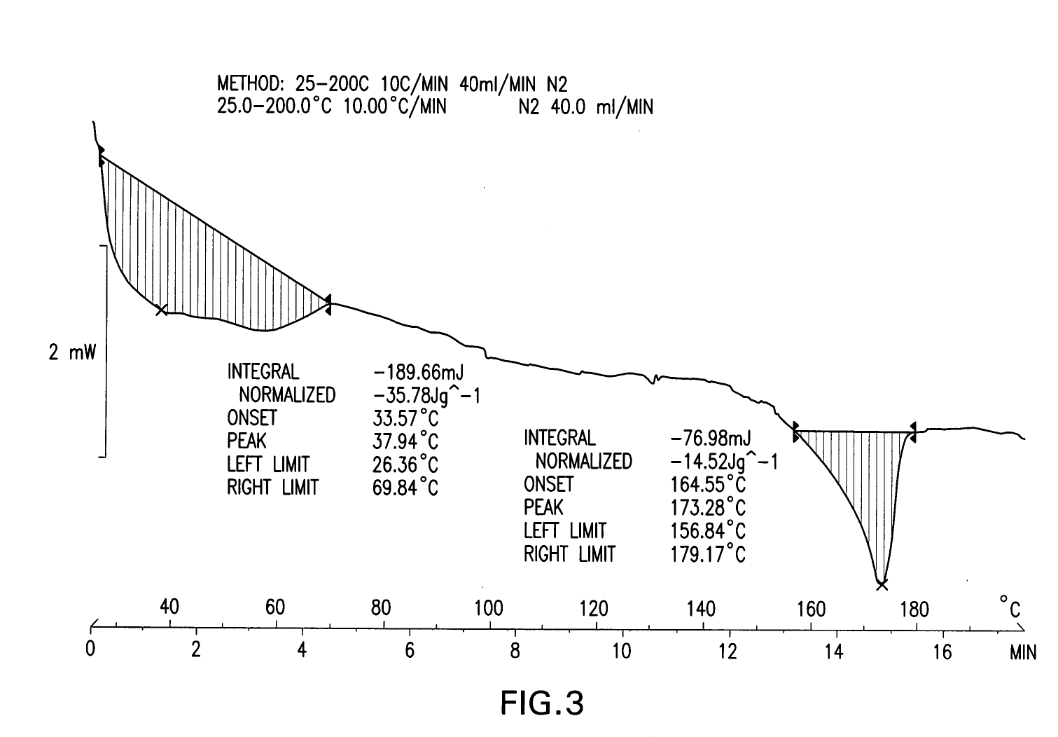

In one embodiment, the present invention encompasses a crystalline form of docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹). The crystalline form may be further characterized by a powder XRD pattern with peaks at about 4.9, 12.5, 13.1, 18.8 and 19.8±0.2 degrees two-theta. The crystalline docetaxel form of the invention has an XRD pattern as substantially depicted in FIG. 1. The crystalline form may be further characterized by a FTIR spectrum with peaks at about 719, 848, 957, 3429 and 3461 (cm⁻¹). The crystalline docetaxel form of the invention has an FTIR spectrum as substantially depicted in FIG. 2. The crystalline form may be further characterized by a DSC thermogram with endothermic peaks at about 30° C. to about 70° C. and 173° C. The crystalline docetaxel form of the invention has a DSC thermogram as substantially depicted in FIG. 3.

The crystalline form of the present invention is anhydrous. The term “anhydrous” refers to a crystalline form having not more than about 1.0% of water or another solvent incorporated into its crystal structure. If the crystal does not undergo a weight loss of more than about 1.0% with TGA, then it is anhydrous.

Another embodiment of the invention encompasses a process for preparing the crystalline docetaxel form of the invention including crystallizing the crystalline docetaxel from a mixture of methyl isobutyl ketone (MIBK) and an organic antisolvent.

Preferably, the process of crystallizing the crystalline docetaxel includes the steps of combining docetaxel in MIBK with an organic antisolvent to obtain the crystalline form. In one embodiment of the invention, docetaxel and MIEBK are combined to form a solution. Preferably, MIBK is present in an amount of about 7 to about 10 volumes (ml per g of docetaxel). Preferably, the mixture of MIBK and docetaxel is heated to a temperature of about 80° C. to about 120° C., more preferably to about 100° C. to facilitate dissolution of the docetaxel.

An organic antisolvent is then preferably added to the solution to obtain a suspension. Preferably, the antisolvent is selected from the group consisting of C₅-C₈ linear and branched alkanes. More preferably, the antisolvent is n-heptane. The antisolvent is preferably added to the solution slowly, such as dropwise, over a period of time of about 30 minutes to about 4 hours. The antisolvent is preferably present in an amount of about 3.5 to about 5 volumes (ml of antisolvent per gram of starting solid).

The suspension may be cooled to increase precipitation and provide a higher yield of the crystalline docetaxel form of the invention. Preferably, the suspension is cooled to a temperature of about 25° C. to about 30° C., more preferably to about 25° C. A mixture of MIBK and n-heptane may optionally be added to the suspension to facilitate recovery of the crystalline docetaxel. Preferably, the crystalline docetaxel is recovered by filtering the suspension and washing it with n-heptane.

Another embodiment of the invention encompasses the crystalline docetaxel form of the invention having a maximal particle size of less than about 300 μm. The term “maximal particle size” refers to a sample containing particles, at least 99% of which have a size equal to or less than the maximal particle size. The particle size of docetaxel crystalline forms may be measured by methods such as: sieves, sedimentation, electrozone sensing (coulter counter), microscopy, and/or Low Angle Laser Light Scattering (LALLS).

Another embodiment of the invention encompasses the crystalline docetaxel form of the invention having less than about 5%, preferably less than about 2%, and more preferably less than about 1% of any other form of docetaxel, particularly docetaxel trihydrate.

Another embodiment of the invention encompasses processes for preparing a crystalline docetaxel form characterized by main X-ray powder diffraction peaks at about 8.0, 11.3, 12.5, 15.5 and 16.9±0.2 degrees two-theta. The crystalline docetaxel maybe further characterized by X-ray powder diffraction peaks at about 4.7, 9.2, 13.9, 20.4 and 23.5 degrees two-theta, ±0.2 degrees two-theta.

The process of the invention for preparing crystalline docetaxel characterized by main X-ray powder diffraction peaks at about 8.0, 11.3, 12.5, 15.5 and 16.9±0.2 degrees two-theta includes precipitating docetaxel from a mixture of a solvent and an organic antisolvent, wherein the solvent is selected from the group consisting of: acetone and ethylacetate, acetone and t-butanol, tetrahydrofuran (THF), ethyl acetate, tert-butanol, ethanol, and mixtures thereof. Preferably, the antisolvent is selected from the group consisting of C₅-C₈ linear and branched alkanes, more preferably n-heptane or n-hexane. In one embodiment, the antisolvent is n-heptane, and the solvent is selected from the group consisting of: acetone/tert-butanol, ethylacetate, ethylacetate/acetone, tetrahydrofuran, and ethanol. In one embodiment, docetaxel and a solvent are combined to form a solution or suspension which is then combined with n-heptane or n-hexane to obtain a reaction mixture.

The solvent is preferably present in an amount of about 3.5 to about 5 volumes (ml per g of docetaxel). Optionally, the reaction mixture of the solvent and docetaxel is heated, preferably to a temperature of about 45° C. to about 65° C., more preferably to a temperature of about 50° C. In one embodiment, when the solvent is a mixture of acetone and tert-butanol, the suspension is not heated.

Preferably the antisolvent is added to a solution or suspension of docetaxel in the solvent. Preferably, the antisolvent is n-heptane. Preferably, the antisolvent is added slowly, such as dropwise, and in an amount of about 3 to about 7 volumes (ml of antisolvent per gram of docetaxel).

Preferably, the reaction mixture is cooled, particularly when preceded by heating, to a temperature of about 0° C. to about 25° C., more preferably to a temperature of about 10° C. Lower temperatures may increase the yield of the obtained form.

Preferably, the crystalline form is recovered by filtration and drying. Preferably, the drying is at a temperature of about 55° C. to about 80° C., under vacuum. Preferably, the obtained crystal form is anhydrous. Optionally, the docetaxel used as a starting material in the process for preparing crystalline docetaxel characterized by main X-ray powder diffraction peaks at about 8.0, 11.3, 12.5, 15.5 and 16.9±0.2 degrees two-theta is crude docetaxel prepared by any of the methods well-known in the art, such as in U.S. Pat. No. 5,808,113.

Docetaxel of defined particle size may be produced by known methods of particle size reduction starting with crystals, powder aggregates and course powder of the new crystalline forms of docetaxel. The principal operations of conventional size reduction are milling of a feedstock material and sorting of the milled material by size.

A fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 μm which may be done using a conventional ball, roller, or hammer mill. One of skill in the art would appreciate that some crystalline forms may undergo a transition to another form during particle size reduction.

The invention also provides a pharmaceutical composition containing a therapeutically effective amount of the crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹), and at least one pharmaceutically acceptable excipient.

Another embodiment of the invention encompasses a process of preparing a pharmaceutical composition including the step of combining crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹), or a solution prepared from the crystalline docetaxel, with a pharmaceutically acceptable carrier.

Yet another embodiment of the invention encompasses a pharmaceutical composition containing a therapeutically effective amount of crystalline docetaxel prepared according to the processes of the present invention, and at least one pharmaceutically acceptable excipient.

Another embodiment of the invention encompasses the use of crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹) in the manufacture of a medicament.

Yet another embodiment of the invention encompasses a method of treating a proliferative disorder, such as cancer, in a mammal, such as a human, including administering to the mammal a pharmaceutically acceptable amount of a pharmaceutical composition containing or prepared from crystalline docetaxel characterized by data selected from the group consisting of: a powder XRD pattern with peaks at about 7.3, 8.8, 13.7, 17.2 and 20.2±0.2 degrees two-theta, and a FTIR pattern with peaks at about 1098, 1165, 1248, 1701 and 1720 (cm⁻¹).

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

The pharmaceutical composition may contain only a single form of docetaxel, or a mixture of various forms of docetaxel, with or without amorphous form. In addition to the active ingredient(s), the pharmaceutical compositions of the invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the invention, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the invention may comprise any of the aforementioned blends and granulates that were described with reference to tableting, however, they are not subjected to a final tableting step.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The dosage used is preferably from about 0.5 mg to about 500 mg of docetaxel, more preferably about 20 to about 100 mg.

### EXAMPLES

General

XRD

X-Ray powder diffraction (XRD) data was obtained using a Scintag X-ray powder diffractometer model X'TRA equipped Cu-tube solid state detector. A round standard aluminum sample holder with rough zero background quartz plate with a cavity of 25 (diameter)×0.5 mm (depth) was used. The scanning parameters included: range: 2-40 degrees two-theta; scan mode: continuous scan; step size: 0.05 deg.; and a rate of 3 deg/min. Copper radiation of 1.5418 Å was used.

Thermal Analysis

DSC thermogram was performed on DSC 821e, Mettler Toledo, with a sample weight of about 3-6 mg, and a heating rate of about 10° C./min at the range 25-250° C. The oven was constantly purged with nitrogen gas at a flow rate of 40 ml/min. Standard 40 μl aluminum crucibles covered by lids with 3 holes were used.

FRIR

FTIR spectroscopy was measured in Perkin-Elmer spectrum One Spectrometer. The samples were analyzed using mineral oil (Nujol). The samples were finely ground and mixed with few drops of mineral oil, and the spectrum was recorded using empty sample chamber as the background. Scanning parameters are: range: 4000-400 cm⁻¹, 16 scans, resolution: 4.0 cm⁻¹.

### Example 1

### Preparation of Crystalline Docetaxel by Crystallization MIBK/n-Heptane

Crude docetaxel (2.8 g, 3.47 mmol) was loaded in a 100 ml reactor, methyl isobutyl ketone (MIBK) was added (21 ml) and the mixture was heated to 80° C. to obtain a clear solution. The n-Heptane (11.7 ml) was added dropwise into the solution and a white solid formed. The mixture was then cooled to 25° C. in 3 h and stirred for 13 h at 25° C.

A preformed mixture of MIBK (4.0 ml) and n-heptane (2.2 ml) was added, obtaining an easily stirrable suspension. The suspension was filtered on gooch P3 and washed with n-heptane (14 ml) obtaining docetaxel (2.13 g) as a white solid (76% yield), having the PXRD as depicted in FIG. 1.

### Example 2

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Acetone/tert-Butanol and n-Heptane

Crude docetaxel (3.0 g, 3.7 mmol) was loaded in a 100 ml round-bottomed flask and dissolved at 25° C. in acetone (24 ml) and tert-butanol (3 ml). The n-Heptane (30 ml) was then slowly added in 15 min and, after stirring at 25° C. for 1 h, the suspension was filtered on gooch P3, washed twice with n-heptane (10 ml) and dried at 55° C. under vacuum for 16 h, obtaining docetaxel (2.80 g) as a white solid with 93.0% yield and 99.6% purity.

### Example 3

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Ethyl Acetate and n-Heptane

5 g of crude docetaxel (99.7% purity) was loaded into a round-bottom glass flask. EtOAc (5 vol, 25 mL) was added to the solid and the suspension was heated up to 50° C. to obtain a solution. Maintaining the temperature, n-heptane (5 vol, 25 mL) was added dropwise. Precipitation was observed at the end of the addition. The suspension was maintained at this temperature under stirring, for another one hour, and then it was cooled to 25° C. After 1 h at 25° C., the suspension was filtered on a gooch P3 and the solid was dried overnight at 55° C. under vacuum.

From the mother liquor a second crop was obtained. The suspension was filtered on a gooch P3, and the solid dried overnight at 55° C. under vacuum to obtain a solid with 99.7% purity

### Example 4

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Ethyl acetate/acetone and n-Heptane

Crude docetaxel (4.8 g, 99.6% purity) was suspended in 10 vol of 1:1 ethyl acetate/acetone mixture. The suspension was heated up to 50° C. At this temperature a suspension was still observed. To the suspension, maintaining the temperature, 27 vol of n-heptane was added dropwise and the suspension was stirred for further 1 h. After this time the mixture was cooled to 0° C. and filtered on a gooch P3, and the solid dried overnight at 55° C. under vacuum to obtain docetaxel in 90% yield and 99.8% purity.

### Example 5

### Preparation of Crystalline Docetaxel Characterized by Main X-ray Powder Diffraction Peaks at about 8.0 11.3, 12.5, 15.5 and 16.9±0.2 Degrees Two-theta by Crystallization from Tetrahydrofuran and n-Heptane

Crude docetaxel (5.0 g, 9.5% purity) was suspended in 4 volume equivalents of tetrahydrofuran. The suspension was heated up to 50° C. At this temperature a solution is observed. Maintaining the temperature, 6 vol of n-heptane are added dropwise to the solution, forming a suspension, which was stirred for one hour further. After this time the mixture was cooled to 25° C. and filtered on a gooch P3, and the solid dried overnight at 55° C. under vacuum to obtain docetaxel in 96% yield and 99.6% purity.

### Example 6

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Tetrahydrofuran and n-Heptane

Crude docetaxel (4.5 g, 99.5% purity) was suspended in 7 volume equivalents of tetrahydrofuran. The suspension was heated up to 50° C. At this temperature a solution was observed. While maintaining the temperature, 10 vol of n-heptane was added dropwise to the solution to form a suspension, which was stirred for one hour further. After this time the mixture was cooled to 0° C. and filtered on a gooch P3, and the solid dried overnight at 55° C. under vacuum to obtain docetaxel in 90% yield and 99.8% purity

### Example 7

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Ethyl Acetate and n-Heptane

Crude docetaxel (9.0 g, 99.5% purity) was loaded into a round-bottom glass flask. EtOAc (9 vol, 81 mL) was added to the solid and the suspension was heated up to 50° C., to obtain a solution. While maintaining the temperature, n-heptane (9 vol, 81 mL) was added dropwise to the solution. Precipitation was observed at the end of the addition. The suspension was maintained at this temperature, under stirring, for one hour further, and then was cooled to 25° C.

After 1 h at 25° C., the suspension was filtered on a gooch P3, and the solid dried overnight at 55° C. under vacuum to obtain docetaxel in 90% yield and 99.8% purity.

### Example 8

### Preparation of Crystalline Docetaxel Characterized by Main X-ray Powder Diffraction Peaks at about 8.0, 11.3, 12.5, 15.5 and 16.9±0.2 Degrees Two-theta by Crystallization from Ethyl Acetate/acetone and n-Heptane

Crude docetaxel (4.3 g, 99.5% purity) was suspended in 7 volume equivalents of 1:1 ethyl acetate/acetone mixture. The suspension was heated up to 50° C. To the resulting suspension, while maintaining the temperature, 7 volume equivalents of n-heptane was added dropwise and the slurry was stirred for further 1 h. After this time the mixture was cooled to 0° C. and filtered on a gooch P3, and the solid dried overnight at 55° C. under vacuum to obtain docetaxel in 90% yield and 99.8% purity.

### Example 9

### Preparation of Crystalline Docetaxel Characterized by main X-ray Powder Diffraction Peaks at about 8.0, 11.3, 12.5, 15.5 and 16.9±0.2 Degrees Two-theta by Crystallization from Ethanol and n-Heptane

Crude docetaxel (5.0 g, 99.5% purity) was loaded into around-bottomed glass flask. EtOH (2 volumes, 10 ml) was added to the solid and the suspension was heated up to 50° C. Maintaining the temperature, n-heptane (5 vol, 25 mL) was added dropwise to the solution thus obtained. The precipitation was observed at the end of the addition. The suspension was maintained at this temperature, under stirring, for one hour further, and then was cooled down to 0° C. After 1 hour at 0° C., the suspension was filtered off on a gooch P3, and the solid dried overnight at 55° C. under vacuum to obtain docetaxel in 80% yield and 99.6% purity.

What is claimed is:

1. Crystalline docetaxel characterized by data selected from the group consisting of: a powder XRD pattern having peaks at about 7.3, 8.8, 13.7, 17.2 and 20.2±0.2 degrees two-theta, and an FTIR spectrum having peaks at about 1098, 1165, 1248, 1701 and 1720 (cm⁻¹).

2. The crystalline docetaxel of claim 1, further characterized by a powder XRD pattern with peaks at about 4.9, 12.5, 13.1, 18.8 and 19.8±0.2 degrees two-theta.

3. The crystalline docetaxel of claim 1, wherein the crystalline docetaxel is further characterized by a FTIR spectrum having peaks at about 719, 848, 957, 3429 and 3461 (cm⁻¹).

4. The crystalline docetaxel of claim 1, wherein the crystalline docetaxel is further characterized by a DSC thermogram with endothermic peaks at about 30° C. to about 70° C. and 173° C.

5. The crystalline docetaxel of claim 1, wherein the crystalline docetaxel has a maximal particle size of less than about 300 μm.

6. The crystalline docetaxel of claim 1, wherein the crystalline docetaxel is further characterized by an XRD pattern as depicted in FIG. 1.

7. The crystalline docetaxel of claim 1, wherein the crystalline docetaxel is further characterized by an FTIR spectrum as depicted in FIG. 2.

8. The crystalline docetaxel of claim 1, wherein the crystalline docetaxel is further characterized by a DSC thermogram as depicted in FIG. 3.

9. The crystalline docetaxel of claim 1, wherein the crystalline docetaxel is anhydrous.

10. The crystalline docetaxel of claim 1, wherein the crystalline docetaxel is present in a composition having less than about 5% of any other form of docetaxel.

11. The crystalline docetaxel of claim 10, wherein the crystalline docetaxel is present in a composition having less than about 2% of any other form of docetaxel.

12. The crystalline docetaxel of claim 11, wherein the crystalline docetaxel is present in a composition having less than about 1% of any other form of docetaxel.

13. The crystalline docetaxel of claim 1, wherein the crystalline docetaxel is present in a composition having less than about 5% of docetaxel trihydrate.

14. The crystalline docetaxel of claim 13, wherein the crystalline docetaxel is present in a composition having less than about 2% of docetaxel trihydrate.

15. The crystalline docetaxel of claim 14, wherein the crystalline docetaxel is present in a composition having less than about 1% of docetaxel trihydrate.

16. A process for preparing the crystalline docetaxel of claim 1 comprising crystallizing docetaxel from a mixture of methyl isobutyl ketone (MIBK) and organic antisolvent.

17. The process of claim 16, wherein crystallizing comprises the steps of:
a) combining docetaxel and MIBK to obtain a solution;
b) adding an organic antisolvent to the solution to obtain a suspension; and
c) recovering the crystalline docetaxel from the suspension.

18. The process of claim 17, further comprising heating the solution of docetaxel and MIBK.

19. The process of claim 18, wherein heating is to a temperature of about 80° C. to about 120° C.

20. The process of claim 16, wherein the antisolvent is selected from the group consisting of C₅-C₈ linear and branched alkanes.

21. The process of claim 20, wherein the antisolvent is n-heptane.

22. The process of claim 17, wherein the antisolvent is added dropwise.

23. The process of claim 18, further comprising cooling the suspension.

24. The process of claim 23, wherein cooling is to a temperature of about 25° C. to about 30° C.

25. The process of claim 23, further comprising the step of adding a mixture of methyl isobutyl ketone (MIBK) and n-heptane to the suspension after cooling.

26. A process for preparing a crystalline docetaxel form characterized by main X-ray powder diffraction peaks at about 8.0, 11.3, 12.5, 15.5 and 16.9±0.2 degrees two-theta, comprising precipitating the crystalline form from a mixture of a solvent and an organic antisolvent, wherein the solvent is selected from the group consisting of: acetone and ethylacetate, acetone and t-butanol, tetrahydrofuran (THF), ethyl acetate, tert-butanol, ethanol, and mixtures thereof.

27. The process of claim 26, wherein the process comprises:
a) combining docetaxel with the solvent to obtain a suspension or solution; and
b) adding an organic antisolvent to precipitate crystalline docetaxel characterized by main X-ray powder diffraction peaks at about 8.0, 11.3, 12.5, 15.5 and 16.9±0.2 degrees two-theta.

28. The process of claim 27, wherein the suspension or solution is heated to a temperature of about 45 to about 65° C.

29. The process of claim 28, wherein the suspension or solution is heated to a temperature of about 50° C.

30. The process of claim 26, wherein the antisolvent is selected from the group consisting of C₅-C₈ linear and branched alkanes.

31. The process of claim 30, wherein the antisolvent is n-heptane or n-hexane.

32. The process of claim 27, wherein the antisolvent is added dropwise.

33. The process of claim 28, further comprising cooling the suspension or solution after adding the antisolvent.

34. The process of claim 33, wherein cooling is to a temperature of about 0° C. to about 25° C.

35. The process of claim 27, further comprising recovering the crystalline docetaxel.

36. The process of claim 35, wherein the crystalline docetaxel is recovered by filtration and drying.

37. The process of claim 36, wherein drying is carried out at a temperature of about 55° C. under vacuum.

38. The process of claim 35, wherein the recovered crystalline docetaxel is anhydrous.

39. A pharmaceutical composition comprising a therapeutically effective amount of the crystalline docetaxel of claim 1 and at least one pharmaceutically acceptable excipient.

40. A pharmaceutical composition comprising a therapeutically effective amount of the crystalline docetaxel prepared according to claim 16 or 26, and at least one pharmaceutically acceptable excipient.

41. A process of preparing a pharmaceutical composition comprising the step of combining the crystalline docetaxel of claim 1, or a solution prepared from the crystalline docetaxel of claim 1, with a pharmaceutically acceptable carrier.

42. A method of treating a mammal suffering from a proliferative disorder comprising administering to the mammal the pharmaceutical composition of claim 39.
